(19) United States
(12) Patent Application Publication
(10) Pub. No.: US 2023/0312643 A1
(43) Pub. Date: Oct. 5, 2023

(54) PEPTIDE DERIVATIVE WITH COLLAGENASE INHIBITORY ACTIVITY, AND USE THEREOF

(71) Applicant: SUNMARINE BIOTECH, Jinju-si, Gyeongsangnam-do (KR)

(72) Inventors: Si Hyang PARK, Jinju-si (KR); Su Yeon HWANG, Jinju-si (KR); Jae Eun JUNG, Jinju-si (KR); Ye Ra KIM, Jinju-si (KR)

(73) Assignee: SUNMARINE BIOTECH, Jinju-si, Gyeongsangnam-do (KR)

(21) Appl. No.: 18/025,167

(22) PCT Filed: Dec. 30, 2020
(86) PCT No.: PCT/KR2020/019459

§ 371 Date: Mar. 7, 2023

(30) Foreign Application Priority Data
Sep. 8, 2020 (KR) 10-2020-0114698

(51) Int. Cl. C07K 5/083; A61Q 19/08

(52) U.S. Cl. CPC C07K 5/0806; A61Q 19/08; A23L 33/18

(57) ABSTRACT

The present invention relates to a peptide derivative, having collagenase inhibitory activity, or a use thereof and has the effects of increasing a percutaneous absorption rate, inhibiting collagenase activity, and reducing wrinkles.

### TECHNICAL FIELD

The present invention claims the benefit of the priority of Korean Patent Application No. 10-2020-0114698 filed on Sep. 8, 2020 with the Korean Intellectual Property Office, the content of which is incorporated herein by reference in its entirety.

The present invention relates to a derivative of peptide and a use thereof.

### BACKGROUND ART

Matrix metalloproteinase (MMP) is an enzyme group involved in the degradation of extracellular matrix (ECM) and basement membrane, and is divided into four subfamilies, which are interstitial collagenase, stromelysin, gelatinase, membrane-type MMP (MT-MMP), etc., according to structure and functional properties.

Each MMP includes a specific amino acid sequence, represents a specific cellular and tissue distribution, and hydrolyzes a specific subset of a target matrix protein. MMP often plays an important role in extracellular signaling, extracellular matrix remodeling, and metabolic control. Thus, appropriate regulation of the activity of MMP is important for the normal generation and maintenance of cells and tissues.

Collagenase is a proteolytic enzyme latently stored in neutrophil-specific granules corresponding to a kind of matrix metalloproteinase (MMP). The active form of collagenase mediates a variety of diseases and disorders in mammals. These diseases and disorders include, but are not limited to, bone resorption diseases such as osteoporosis and metastatic bone marrow cancer, corneal ulcers, periodontal diseases, inflammatory joint diseases, skin inflammatory diseases and wounds, and burns.

Thus, attempts have been continuously made to treat collagenase-mediated diseases or disorders by using a substance which inhibits excessive activity of collagenase (MMP-1) having the above properties. In addition, this collagenase has been reported as a major factor in photoaging because the collagenase can remarkably reduce collagen due to increased activity on the skin during a single UV irradiation, and thus many studies have been conducted for reducing wrinkles in the cosmetics industry, focusing on the inhibition of collagenase activity.

### DISCLOSURE

### Technical Problem

An object of the present invention is to provide a peptide derivative which inhibits collagenase activity.

Still another object of the present invention is to provide a cosmetic composition including the peptide derivative as an active ingredient.

Still another object of the present invention is to provide a pharmaceutical composition including the peptide derivative as an active ingredient.

Still another object of the present invention is to provide a health functional food composition including the peptide derivative as an active ingredient.

### Technical Solution

According to one embodiment of the present invention, there may be provided a peptide derivative represented by formula 1 below or a pharmaceutically acceptable salt thereof.

R₂-L-R₂ [Formula 1]

wherein,

L is Gly-Pro-Asn (GPN) or Pro-Gly-Asn (PGN),

R₁ is hydrogen, C₁-C₆ alkyl group, C₁-C₆ alkoxy group, palmitoyl group, lauroyl group, myristoyl group, stearoyl group, arachidoyl group or linoleoyl group,

R₂ is a hydroxy group, C₁-C₆ alkyl group or C₁-C₆ alkoxy group, and when R₁ is hydrogen, R₂ is not a hydroxy group.

According to one embodiment of the present invention, there may be provided a peptide derivative or a pharmaceutically acceptable salt thereof, in which above formula 1 is represented by formula 2 and formula 3.

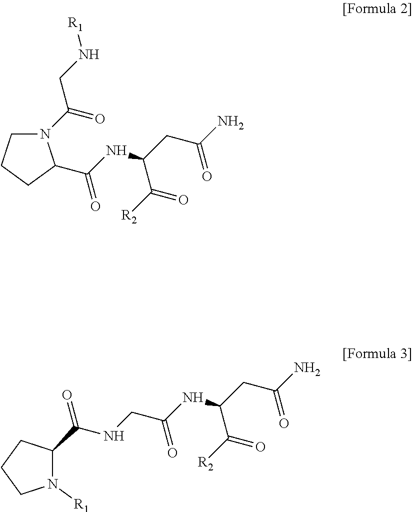

In above formula 2 or 3,

R₁ is hydrogen, CH₃ or a palmitoyl group, and R₂ is a hydroxy group, C₃H₇ or O—C₃H₇, and when R₁ is hydrogen, R₂ is not a hydroxy group.

According to one embodiment of the present invention, there may be provided a peptide derivative or a pharmaceutically acceptable salt thereof, in which above formula 2 is any one of formulas 4 to 8.

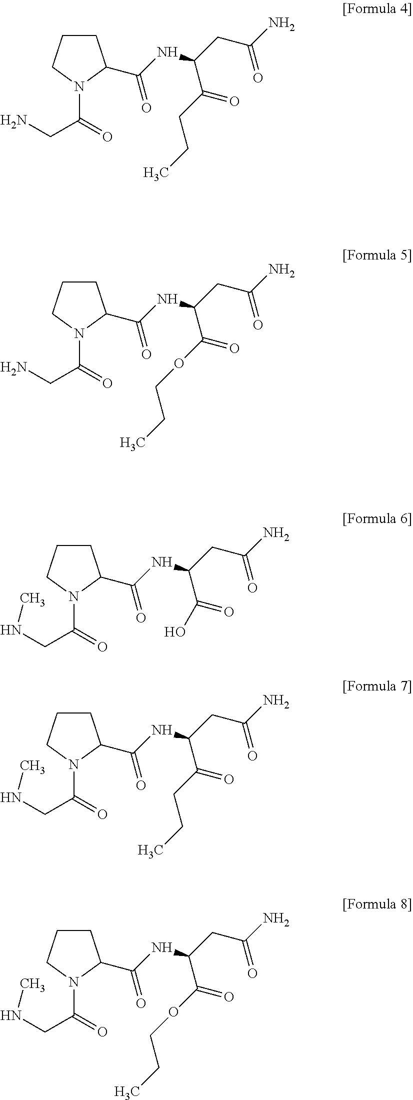 [SMILES: CCCC(=O)[C@H](CC(N)=O)NC(=O)C1CCCN1C(=O)CN ; CCCOC(=O)[C@H](CC(N)=O)NC(=O)C1CCCN1C(=O)CN ; CNCC(=O)N1CCCC1C(=O)N[C@@H](CC(N)=O)C(=O)O ; CCCC(=O)[C@H](CC(N)=O)NC(=O)C1CCCN1C(=O)CNC ; CCCOC(=O)[C@H](CC(N)=O)NC(=O)C1CCCN1C(=O)CNC]

According to one embodiment of the present invention, there may be provided a peptide derivative or a pharmaceutically acceptable salt thereof, in which above formula 3 is any one of formulas 9 to 13.

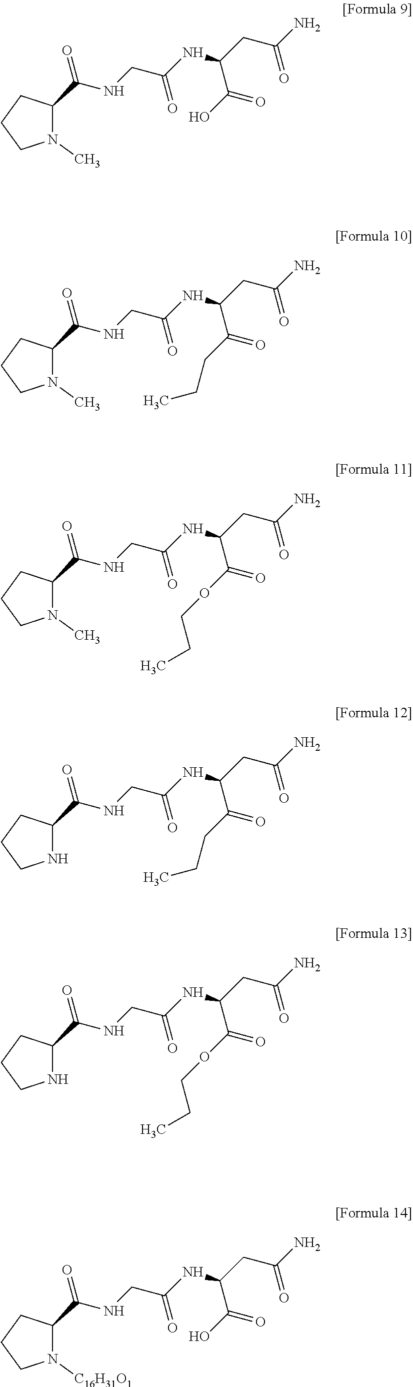

According to one embodiment of the present invention, there may be provided a cosmetic composition including the peptide derivative; or the pharmaceutically acceptable salt thereof as an active ingredient.

According to one embodiment of the present invention, the cosmetic composition may be a composition for alleviating skin aging, which inhibits the activity and production of collagenase.

According to one embodiment of the present invention, the cosmetic composition may be a composition for reducing skin wrinkles or improving skin elasticity.

According to one embodiment of the present invention, the cosmetic composition may be a composition having improved percutaneous absorbing capacity.

According to one embodiment of the present invention, there may be provided a pharmaceutical composition which includes: a peptide derivative; or a pharmaceutically acceptable salt thereof as an active ingredient and inhibits the activity and production of collagenase.

According to one embodiment of the present invention, there may be provided a health functional food composition which includes: a peptide derivative; or a pharmaceutically acceptable salt thereof as an active ingredient and inhibits the activity and production of collagenase.

According to one embodiment of the present invention, there may be provided the use of the peptide derivative; or the pharmaceutically acceptable salt thereof for preparing a drug which inhibits the activity and production of collagenase.

### Advantageous Effects

A peptide derivative according to the present invention can effectively inhibit collagenase activity, and thus the composition including the same as an active ingredient can be advantageously used for preventing, alleviating or treating diseases related to collagenase activity.

The peptide derivative of the present invention is effective in reducing wrinkles, and thus can be widely used as a material in various fields such as the drug industry, the food industry and the cosmetic industry.

In addition, the peptide derivative of the present invention has the advantage of increasing a percutaneous absorption rate and having no cytotoxicity, and thus can be safely used.

### DESCRIPTION OF DRAWINGS

FIG. 1 shows a docking profile of unmodified ligands [1CGL, GPN, PGN (top)] and modified ligands [1CGL, Me-GPN-propyl, Me-PGN-propyl (bottom)].

FIG. 2 shows an analysis result of PGN.

FIG. 3 shows an analysis result of PGN-OPr.

FIG. 4 is a graph comparing collagenase inhibitory activities of PGN and a derivative thereof.

FIG. 5 is a graph comparing collagenase inhibitory activities of PGN.

FIG. 6 is a graph comparing collagenase inhibitory activities of PGN-OPr.

FIG. 7 is a graph comparing elastase inhibitory activities of PGN and a derivative thereof.

FIG. 8 is a graph comparing elastase inhibitory activities of PGN.

FIG. 9 is a graph comparing elastase inhibitory activities of PGN-OPr.

FIG. 10 is a graph comparing gelatinase inhibitory activities of PGN and a derivative thereof.

FIG. 11 is a graph showing the results of HaCaT cytotoxicity tests of PGN, Me-PGN, PGN-OPr and Pal-PGN.

FIG. 12 is a graph showing the results of HS68 cytotoxicity tests, which are skin dermal cells of PGN, Me-PGN, PGN-OPr, and Pal-PGN.

### BEST MODE

Hereinafter, the present invention will be described in more detail.

The following specific functional descriptions are merely exemplified to describe an embodiment according to the concept of the present invention, and the embodiments according to the concept of the present invention may be implemented in various forms and should not be construed as being limited to the embodiments described in the present specification.

Since embodiments according to the concept of the present invention may have various modifications and various forms, specific embodiments will be described in detail herein. However, this is not intended to limit the embodiments according to the concept of the present invention to specific disclosed forms, and it should be understood that the present invention includes all modifications, equivalents, and replacements included in the spirit and technical scope of the present invention.

Terms used in the present application are used only to describe a certain embodiment and are not intended to limit the present invention. The terms of a singular form may include plural forms unless otherwise specified.

All the terms used herein including technical or scientific terms have the same meaning as commonly understood by those ordinary skilled in the art, to which the present invention pertains, unless defined otherwise. Such terms as those defined in a generally used dictionary are to be interpreted to have the meanings equal to the contextual meanings in the relevant art, and are not to be interpreted to have ideal or excessively formal meanings, unless clearly defined in the present specification.

As used herein, the term “alkoxy” may refer to an —O-alkyl group. Examples of the alkoxy group may include methoxy, ethoxy, propoxy (e.g., n-propoxy and isopropoxy), t-butoxy and the like.

As used herein, the term “alkyl” may refer to a linear or branched saturated hydrocarbon group. Examples of the alkyl group may include methyl (Me), ethyl (Et), and propyl (e.g., n-propyl and isopropyl), butyl (e.g. n-butyl, isobutyl, t-butyl), pentyl (e.g. n Pentyl, isopentyl, neopentyl), and the like.

As used herein, the term “collagenase” may refer to a proteolytic enzyme which is latently stored in neutrophil-specific granules corresponding to a kind of matrix metalloproteinase (MMP), and the active type of collagenase is known to mediate various diseases and disorders in mammals.

In various parts of the present specification, substituents of the compound of the present invention may be described as groups or ranges. Specifically, the present invention may be intended to include respective and all individual sub-combinations of members of these groups and ranges. For example, the term “C₁-C₆ alkyl” may be intended to describe methyl, ethyl, C₃ alkyl, C₄ alkyl, C₅ alkyl, and C₆ alkyl, individually.

The present invention may include pharmaceutically acceptable salts of the compound described in the present specification. As used herein, the term “pharmaceutically acceptable salt” may refer to a derivative in which a parent compound is modified by converting an acid or basic residue into a salt form thereof. Examples of the pharmaceutically acceptable salt may include inorganic or organic acid salts of basic residues, for example, amines; alkali or organic salts of acidic residues, for example, carboxylic acid, etc. The pharmaceutically acceptable salts of the present invention may include conventional non-toxic salts or quaternary ammonium salts of parent compounds formed from non-toxic inorganic or organic acids. The pharmaceutically acceptable salts of the present invention may be synthesized from parent compounds including basic or acidic residues by using conventional chemical methods.

In the present invention, the term “including as an active ingredient” may refer to containing an effective amount enough for alleviating skin aging, ameliorating skin wrinkles or elasticity, and improving percutaneous absorption as a cosmetic composition. A content of the peptide derivative in the composition may be included in the range of 0.001 to 10 wt % based on the total composition.

In the present invention, the term “skin improvement” may be a concept including improvement of a skin condition, and may include prevention of skin aging including reduction of skin wrinkles, improvement of elasticity, and enhancement of a whitening function.

The peptide of the present invention may be contained in an amount of preferably 0.0001 to 1.0 wt %, more preferably 0.001 to 1.0 wt %, and most preferably 0.001 to 0.01 wt % based on the total weight of the cosmetic composition, but is not limited thereto.

The cosmetic composition according to the present invention may include ingredients conventionally used in the cosmetic composition in addition to the derivative of peptide as an active ingredient, and may include, for example, conventional adjuvants such as stabilizers, solubilizers, vitamins, pigments, and perfumes, and carriers.

In addition, the cosmetic composition according to the present invention may be prepared in the form of a formulation generally known in the art, for example, in the form of an emulsion formulation, a solubilized formulation, or the like. Examples of the emulsion formulation may include nourishing lotion, cream, essence, and the like, and examples of the solubilized formulation may include softening lotion. In addition, the cosmetic composition of the present invention may be prepared in the form of an adjuvant commonly used in the field of dermatology to be applied topically or systemically by containing a medium or a base which is scientifically acceptable to the skin in addition to cosmetics.

A formulation suitable for cosmetics may be provided, for example, in the form of solution, gel, solid or paste anhydrous product, emulsion obtained by dispersing an oil phase in an aqueous phase, suspension, ionic and nonionic vesicular dispersion such as microemulsion, microcapsule, microgranulocyte or liposome, cream, skin, lotion, powder, ointment, spray or conical stick. Further, the formulation may also be prepared in the form of a foam or an aerosol composition further containing a compressed propellant.

The cosmetic composition may include an acceptable carrier in a cosmetic preparation. Here, the term “acceptable carrier in the cosmetic preparation” may refer to a compound or composition which may be included in the cosmetic preparation and is already known and used, or a compound or composition to be developed in the future, which has no more toxicity, instability, or irritation than the human body can adapt to when coming into contact with skin.

The carrier may be included in the composition for skin external application of the present invention in an amount of about 1 wt % to about 99.99 wt %, preferably about 90 wt % to about 99.99 wt % based on the total weight of the composition. However, since the above ratio varies depending on the formulation as described later in which the composition for skin external application of the present invention is prepared and depending on a specific application site thereof (face, neck, etc.) or a preferred application amount thereof, etc., the above ratio is not to be understood as limiting the scope of the present invention in any aspect.

Examples of the carrier may include an alcohol, oil, surfactant, fatty acid, silicone oil, wetting agent, moisturizer, viscosity modifier, emulsion, stabilizer, ultraviolet scattering agent, ultraviolet absorber, coloring agent, fragrance, and the like. Since the compounds/compositions which may be used as the alcohol, oil, surfactant, fatty acid, silicone oil, wetting agent, moisturizer, viscosity modifier, emulsion, stabilizer, ultraviolet scattering agent, ultraviolet absorber, coloring agent, fragrance, etc. are already known in the art, those skilled in the art may select and use appropriate materials/compositions. Further, an organic/inorganic sunscreen agent known in the related art, a natural substance known to have a sunscreen function, and the like may be additionally included.

In addition, the cosmetic composition of the present invention may include glycerin, butylene glycol, propylene glycol, polyoxyethylene hydrogenated castor oil, ethanol, triethanolamine, and the like, in addition to the peptide derivative, and may include a small amount of preservatives, fragrance, coloring agent, purified water, and the like, as necessary.

Further, in addition to the derivative of peptide, the cosmetic composition of the present invention may further contain fat materials, organic solvent, dissolvent, thickening and gelling agents, softener, antioxidant, suspending agent, stabilizer, foaming agent, flavoring agent, surfactant, water, ion type or non-ion type emulsifier, filler, sequestering and chelating agents, preservatives, vitamin, blocking agent, humectant, essential oil, dye, pigment, hydrophilic or lipophilic activator, lipid vesicle, or adjuvants, which are conventionally used in a field of cosmetic science or dermatology, such as any other ingredients conventionally used in cosmetics. And, the above ingredients may be introduced in such an amount that is generally used in the field of dermatology. Products to which the cosmetic composition of the present invention may be added include, for example, cosmetics such as astringent lotion, softening lotion, nourishing lotion, various creams, essence, pack, foundation, etc., cleansing agent, soap, treatment, cosmetic solution, etc.

Specific formulations of the cosmetic composition of the present invention may include formulations such as skin lotion, skin softener, skin toner, astringent, lotion, milky lotion, moisture lotion, nourishing lotion, massage cream, nourishing cream, moisture cream, hand cream, essence, nourishing essence, pack, soap, shampoo, cleansing foam, cleansing lotion, cleansing cream, body lotion, body cleanser, emulsion, pressed powder, loose powder, eye shadow, patch, spray, and the like.

The cosmetic composition of the present invention may be daily used and may also be used for an undefined period. Preferably, the amount of use, the number of uses, and the period of use may be adjusted according to a user's age, skin condition or skin type, and the concentration of peptide.

According to one embodiment of the present invention, there may be provided a pharmaceutical composition which includes: a peptide derivative; or a pharmaceutically acceptable salt thereof as an active ingredient and inhibits the activity and production of collagenase. The pharmaceutical composition may be used for preventing or treating collagenase-mediated diseases. “Collagenase-mediated diseases” may include bone resorption diseases such as osteoporosis and metastatic bone marrow cancer, corneal ulcers, periodontal diseases, inflammatory joint diseases, skin inflammatory diseases and wounds, and burns, but are not limited thereto.

The pharmaceutical composition of the present invention may be prepared by using a pharmaceutically suitable and physiologically acceptable adjuvant in addition to the active ingredient, and may use an excipient, disintegrant, sweetener, binder, coating agent, swelling agent, lubricant, glidant, flavoring agent, or the like as the glidant.

The pharmaceutical composition may be preferably formulated into a pharmaceutical composition including one or more pharmaceutically acceptable carriers in addition to the above-described active ingredients for administration.

The pharmaceutical composition may have a formulation type of a granule, powder, tablet, coated tablet, capsule, suppository, liquid, syrup, juice, suspension, emulsion, drop, injectable liquid or the like. For example, for formulation in the form of tablet or capsule, the active ingredient may be combined with an oral, non-toxic pharmaceutically acceptable inert carrier such as ethanol, glycerol, water, etc.

In addition, a suitable binder, lubricant, disintegrant and coloring agent may also be included in the mixture, if desired or required. The suitable binder may include starch, gelatin, natural sugar such as glucose or beta-lactose, corn, sweetener, natural and synthetic gums such as acacia, tragacanth or sodium oleate, sodium stearate, magnesium stearate, sodium benzoate, sodium acetate, sodium chloride, and the like, but is not limited thereto.

The disintegrant may include starch, methyl cellulose, agar, bentonite, xanthan gum, and the like, but is not limited thereto. In the composition formulated into a liquid solution, the pharmaceutically acceptable carrier used herein may include saline solution, sterilized water, Ringer's solution, buffered saline, albumin injection solution, dextrose solution, maltodextrin solution, glycerol, ethanol and a mixture of at least one component thereof, and other conventional additives such as antioxidants, buffer solutions, bacteriostatic agents, etc., may be added thereto, if needed. In addition, a diluent, dispersant, surfactant, binder, and lubricant may be additionally added.

The pharmaceutical composition may be formulated into a conventional pharmaceutical formulation known in the art. The pharmaceutical composition may be formulated into a dosage foam of an oral administration formulation, injectable formulation, suppository formulation, transdermal administration formulation, and a nasal administration formulation, and then administered. For example, the formulation may be a formulation for oral administration such as a liquid, suspension, powder, granule, tablet, capsule, pill, skin external application, or extract.

In still another embodiment of the present invention, the present invention may provide a health functional food composition which includes the peptide derivative as an active ingredient and inhibits collagenase production or activity.

The health functional food composition of the present invention may be prepared and processed in the form of tablet, capsule, powder, granule, liquid, pill, etc.

In the present invention, the “health functional food” may refer to a food prepared and processed by using a raw material or component, which has functionality useful for the human body, and mean taking such food for the purpose of adjusting nutrients with regard to structures and functions of the human body or obtaining an effect valuable for health uses such as a physiological action, etc.

The health food composition of the present invention may contain conventional food additives, and whether a certain item is suitable as the “food additives” or not is decided on the basis of specifications and standards on such item according to the general rules, other general testing methods and the like of the Food Additives Code approved by the Ministry of Food and Drug Safety, unless there are other regulations.

As items listed on said “Food Additives Code,” there may be, for example, chemical compounds such as ketones, glycine, potassium citrate, nicotinic acid, cinnamic acid, etc.; natural additives such as persimmon color, licorice extract, crystalline cellulose, kaoliang color, guar gum, etc.; and mixed formulations such as L-sodium glutamate formulation, alkali additives for noodles, preservatives formulation, tar color formulation, etc.

For example, the health functional food in the form of a tablet may be prepared by granulating a mixture of a peptide derivative (a peptide having one amino acid sequence selected from the group consisting of GPN, AFN, and PGN), which is an active ingredient of the present invention, with an excipient, binder, disintegrant, and other additives by a conventional method, and then compression-molding the granulated mixture by adding a lubricant, or the like, or directly compression-molding the mixture. In addition, the health functional food in the form of a tablet may contain bitters or the like, if necessary.

The health functional food including the peptide derivative of the present invention as an active ingredient may be effective in ameliorating collagenase-mediated diseases since collagenase activity may be significantly suppressed as confirmed in the following Examples.

If the health functional food composition of the present invention is used as food additives, the peptide derivative may be added as it is, or may be used along with other foods or food ingredients, and may be appropriately used according to a conventional method.

A mixed amount of the active ingredients may be appropriately determined according to the purpose of use (prevention, health, or therapeutic treatment). In general, when preparing food or beverage, the composition of the present invention may be added in an amount of 15 parts by weight or less, preferably 10 parts by weight or less, based on a raw material. However, in the case of a long-term intake for health and hygiene purposes or health control purposes, the amount may be equal to or less than the above range and the active ingredient may be used in an amount equal to or greater than the above range since there is no problem in terms of safety.

A type of said food is not particularly limited. An example of food, to which the extract may be added, may include meat, sausage, bread, chocolate, candy, snack, confectionery, pizza, instant noodle, other noodle, chewing gum, dairy products including ice cream, various types of soup, beverage, tea, health drink, alcohol beverage, vitamin complex and the like, and include all the health foods in a conventional sense.

When the composition of the present invention is used as a health drink, the composition may contain various flavoring agents, natural carbohydrates or the like as additional ingredients like a typical drink. The above-described natural carbohydrates may be monosaccharides such as glucose and fructose, disaccharides such as maltose and sucrose, polysaccharides such as dextrin and cyclodextrin, and sugar alcohols such as xylitol, sorbitol, erythritol, etc. Examples of the sweetener may include natural sweeteners such as thaumatin and stevia extract, synthetic sweeteners such as saccharin and aspartame, and the like. The ratio of the natural carbohydrate may be generally about 0.01 to 0.04 g, preferably about 0.02 to 0.03 g per 100 g of the composition of the present invention. Besides the above, the composition of the present invention may contain various nutritional supplements, vitamin, electrolyte, flavoring agent, coloring agent, pectic acid and salt thereof, alginic acid and salt thereof, organic acid, protective colloidal thickener, pH adjusting agent, stabilizer, preservative, glycerin, alcohol, carbonation agent used in carbonated beverage, etc. Besides, the composition of the present invention may contain natural fruit juice and pulp for preparing natural fruit juice, fruit juice beverage and vegetable based beverage. The ingredients may be used independently or in combination. The ratio of the additive is not significantly important, but the composition of the present invention may be generally selected in the range of 0.01 to 0.1 parts by weight per 100 parts by weight.

Hereinafter, the present application will be described in more detail through Preparation Examples, Examples, Comparative Examples, and Experimental Examples, but the following Examples are only for helping understanding of the present application, and do not limit the scope of the present application.

### <Preparation Example 1> Preparation of PGN(Pro-Gly-Asn)-OPr (Example 1

1. Add 2-chlorotrityl chloride resin to a reactor.

2. Add dichloromethane (DCM) to the reactor and make the resin swollen at room temperature for 20 minutes.

3. Discharge the DCM.

4. Dissolve Fmoc-Asn (Trt)-OH (using 4 equivalents for resin (substitution rate), DIPEA (N,N-diisopropylethylamine; 4.4 equivalents for resin (substitution rate)) in DCM.

5. Pour the DCM solution of Fmoc-Asn (Trt)-OH/DIPEA into the reactor containing the resin and then stir at room temperature for four hours.

6. Remove the reaction solvent, add DCM/MeOH/DIPEA (17:2:1) thereto, and stir for 10 minutes.

7. Remove the solution, add DCM/MeOH/DIPEA (17:2:1) thereto, and stir for 10 minutes.

8. Remove the solution and wash twice with DMF.

9. Add 20% piperidine/DMF to the resin in the reactor and stir twice for 15 minutes.

10. Remove the solution and wash the resin six times in total with DMF.

11. Dissolve Fmoc-Gly-OH (using 3 equivalents for resin), HOBt (1-hydroxybenzotriazole; using 3.3 equivalents for resin), DIC (using N,N-diisopropylcarbodiimide: 3.3 equivalents for resin) in DMF.

12. Add the dissolved Fmoc-Gly-OH/HOBt and DIC in the reactor containing resin and stir at room temperature for four hours.

13. Remove the reaction solvent and wash twice with DMF.

14. Add 20% piperidine/DMF to the resin and stir twice for 15 minutes.

15. Wash six times with DMF.

16. Repeat the above steps 11 to 15 by using the required amino acid (Fmoc-Pro-OH) in the sequence. (Order: PGN)

17. Finally, remove Fmoc and then protect an N-terminus with Boc by using (Boc)2O/DIPEA.

18. Upon final coupling, wash the resin with DMF (three times) and DCM (three times), respectively.

19. Remove the protected peptide (Boc-Pro-Gly-Asn (Trt)-OH) from the resin with 3% TFA/DCM solution and crystallize with ethyl ether so as to obtain the protected peptide.

20. React the protected peptide with n-propyl alcohol in the presence of a catalyst, so as to obtain Boc-Pro-Gly-Asn (Trt)-OPr.

21. Treat the resulting protected Boc-Pro-Gly-Asn (Trt)-OPr with trifluoroacetic acid (TFA), water and triisopropylsilane (TIS) (95/2.5/2.5) cocktail, so as to remove the protecting group.

22. Pour the cooled ethyl ether into the reaction solution to crystallize, and then obtain the resulting peptide after centrifugation. 23. Isolate and purify crude peptide by a reverse phase (RP)-HPLC system, so as to obtain a peptide solution.

24. Obtain the final product after lyophilization and confirmed with MALDI-TOF MASS.

### <Preparation Example 2> Preparation of PGN (Pro-Gly-Asn) (Comparative Example 3

1. Add 2-chlorotrityl chloride resin to a reactor.

2. Add dichloromethane (DCM) to the reactor and make the resin swollen at room temperature for 20 minutes.

3. Discharge the DCM.

4. Dissolve Fmoc-Asn (Trt)-OH (using 4 equivalents for resin (substitution rate), DIPEA (N,N-diisopropylethylamine; 4.4 equivalents for resin (substitution rate)) in DCM.

5. Pour the DCM solution of Fmoc-Asn (Trt)-OH/DIPEA into the reactor containing the resin and then stir at room temperature for four hours.

6. Remove the reaction solvent, add DCM/MeOH/DIPEA (17:2:1) thereto, and stir for 10 minutes.

7. Remove the solution, add DCM/MeOH/DIPEA (17:2:1) thereto, and stir for 10 minutes.

8. Remove the solution and wash twice with DMF.

9. Add 20% piperidine/DMF to the resin in the reactor and stir twice for 15 minutes.

10. Remove the solution and wash the resin six times in total with DMF.

11. Dissolve Fmoc-Gly-OH (using 3 equivalents for resin), HOBt (1-hydroxybenzotriazole; using 3.3 equivalents for resin), DIC (using N,N-diisopropylcarbodiimide: 3.3 equivalents for resin) in DMF.

12. Add the dissolved Fmoc-Gly-OH/HOBt and DIC in the reactor containing resin and stir at room temperature for four hours.

13. Remove the reaction solvent and wash twice with DMF.

14. Add 20% piperidine/DMF to the resin and stir twice for 15 minutes.

15. Wash six times with DMF.

16. Repeat the above steps 13 to 15 by using the required amino acid (Fmoc-Pro-OH) in the sequence. (Order: PGN)

17. Upon final coupling, wash the resin with DMF (three times) and DCM (three times), respectively, and perform drying.

18. Treat the resin with trifluoroacetic acid (TFA), water, and triisopropylsilane (TIS) cocktail, so as to deprotect the protecting group of the peptide.

19. Filter off the resin, crystallize the filtrate with pre-cooled ether, and then obtain the resulting peptide after centrifugation.

20. Isolate and purify crude peptide by a reverse phase (RP)-HPLC system, so as to obtain a peptide solution. Obtain the final product after lyophilization and confirmed with MALDI-TOF MASS.

### <Experimental Example 1> Molecular Modeling Experiment on Binding Affinity of Active Substance and Collagenase Inhibitory Activity

Molecular modeling was conducted through a computer simulation program.

Target structure: MMP1 protein (pdb1cgl)

Inhibitor: pdb1cgl_ligand (ligand binding to pdb, Ki=135 nM), Gly-Pro-Asn, Pro-Gly-Asn

Software: SYBYL-X 2.1.1, Surflex-Dock2.7

The goldscores calculated by performing molecular docking on four ligands, 1CGL, 966C, 2TCL, and 1HFC were 85.6, 80.4, 70.2, and 68.3, respectively, and thus 1CGL was used as a molecular model for collagenase inhibitor molecular docking.

Through a computer simulation program, a study was conducted on how to promote collagenase enzyme inhibitory activity and percutaneous absorption during peptide modification.

Table 1 below shows the Surflex-Dock scores of pdb 1CGL, GPN, PGN and modified peptides and the structure of the compounds.

Referring to table 1, the binding affinity between GPN and PGN was about 51.8% and 61.5%, respectively, compared to 1cgl ligand, and PGN was about 10% higher than GPN. Meanwhile, the binding affinity of Me-GPn-propyl and Me-PGn-propyl obtained by modifying the N- and C-termini of GPN and PGN with a methyl group and an n-propyl group, respectively, was 74.7% and 90.1%, respectively, which were increased by 21.9% and 28.6%, respectively, compared to before the modification. In addition, it was found that similarity increases according to the modification. Total score is a value indicating the degree of binding affinity of a docked structure including a crash and a polar interaction in the unit of pKd, and the larger the value, the higher the binding affinity. And, the similarity is a value comparing the similarity with the structure already bonded to the 1cgl, and means that the closer to 1, the more similar. The result of such docking means that the addition of an appropriate modification residue through a docking simulation of computer chemistry may increase the binding affinity and similarity.

TABLE 1

| | | Total | | | |
|---|---|---|---|---|---|
| Ligand | Structure | score | Crash | Polar | Similarity |
| PDB 1CGL (Comparative Example 1) | 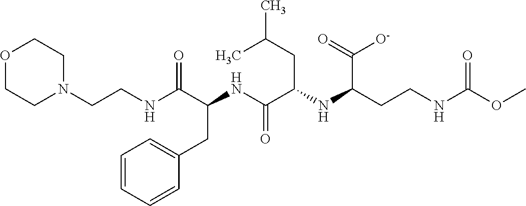 [SMILES: COC(=O)NCC[C@@H](N[C@@H](CC(C)C)C(=O)N[C@@H](Cc1ccccc1)C(=O)NCCN1CCOCC1)C(=O)[O-]] | 13.5281 | −2.592 | 7.2835 | 0.849 |
| GPN (Comparative Example 2) | 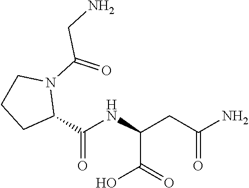 [SMILES: NCC(=O)N1CCC[C@H]1C(=O)N[C@@H](CC(N)=O)C(=O)O] | 7.0033 | −1.1947 | 5.0882 | 0.301 |
| PGN (Comparative Example 3) | 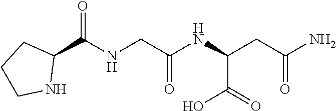 [SMILES: NC(=O)C[C@H](NC(=O)CNC(=O)[C@@H]1CCCN1)C(=O)O] | 8.3245 | −1.4383 | 5.342 | 0.322 |
| Me-GPN- Propyl (Example 6) | 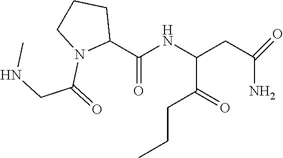 [SMILES: CCCC(=O)C(CC(N)=O)NC(=O)C1CCCN1C(=O)CNC] | 10.1008 | −2.1726 | 4.8227 | 0.370 |
| Me-PGN- Propyl (Example 7) | 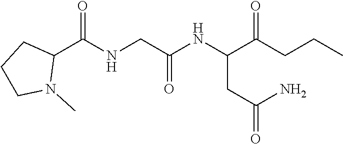 [SMILES: CCCC(=O)C(CC(N)=O)NC(=O)CNC(=O)C1CCCN1C] | 12.1821 | −1.3768 | 5.591 | 0.407 |

Table 2 shows the consensus scores of pdb 1CGL, GPN, PGN and modified peptides. Referring to table 2, as a result of calculating the binding affinity between the pdb ligand and the peptide by using an additional scoring function for calculating the protein-ligand binding affinity in addition to the Surflex-Dock score, it was found that total score is dominant in pdb1cgl_ligand, and PMF_score is dominant in PGN. In the case of GPN, it was found that PMF_score and ChemScore are superior to pdb1cgl_ligand, but show lower binding affinity compared to PGN. Considering CSCOR, it was found that PDB 1cgl has the best in one out of five scoring functions, while Me-PGn-propyl has the predominance of D score and Chemscore. It was found that the binding affinity of pdb1cgl_ligand is remarkably high in terms of the total score only. However, when compared with the overall consensus score, a binding affinity similar to or higher than that of pdb1cgl_ligand may be expected, particularly in the modified GPN and PGN, too.

TABLE 2

| | Total | D | PMF | G | | |
|---|---|---|---|---|---|---|
| Ligand | score | score | score | score | Chemscore | CSCORE |
| PDB 1CGL | 13.5281 | −92.6999 | −50.8229 | −194.4065 | −12.6840 | 1 |
| (Comparative | | | | | | |
| Example 1) | | | | | | |
| GPN | 7.0033 | −84.1044 | −84.8170 | −162.8013 | −17.0717 | 0 |
| (Comparative | | | | | | |
| Example 2) | | | | | | |
| PGN | 8.3245 | −91.8287 | −85.2727 | −203.6772 | −19.0459 | 1 |
| (Comparative | | | | | | |
| Example 3) | | | | | | |
| Me-GPN-Propyl | 10.1008 | −115.4355 | −15.5692 | −255.6655 | −28.3926 | 1 |
| (Example 6) | | | | | | |
| Me-PGN-Propyl | 12.1821 | −118.6310 | −51.0662 | −248.6420 | −32.0104 | 2 |
| (Example 7) | | | | | | |

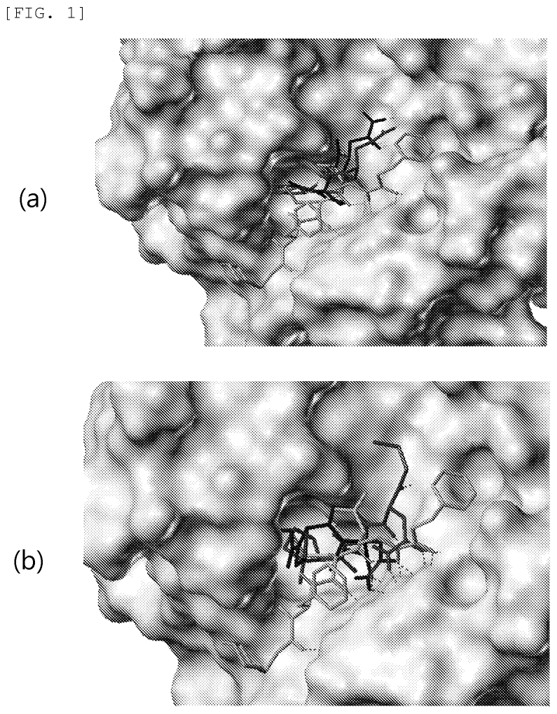

FIG. 1 shows a docking profile of unmodified ligands [1CGL, GPN, PGN (top)] and modified ligands [1CGL, Me-GPN-propyl, Me-PGN-propyl (bottom)], in which green, blue and red represent 1CGL ligand, GPN and PGN, respectively.

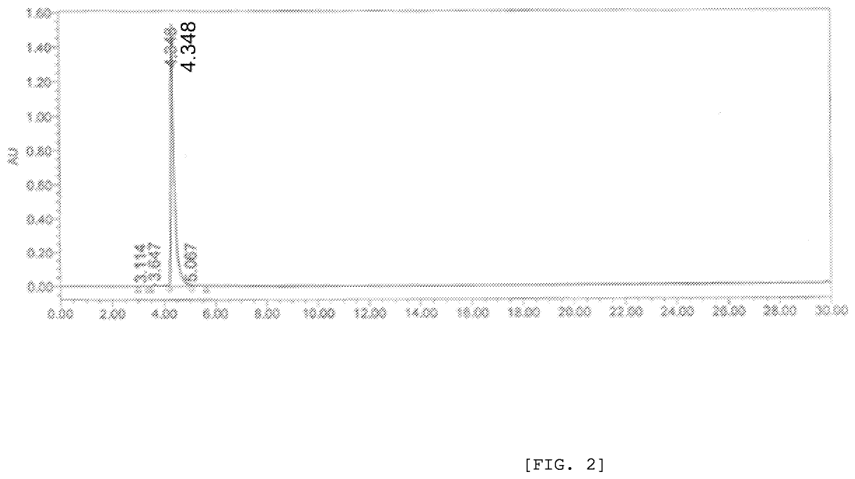

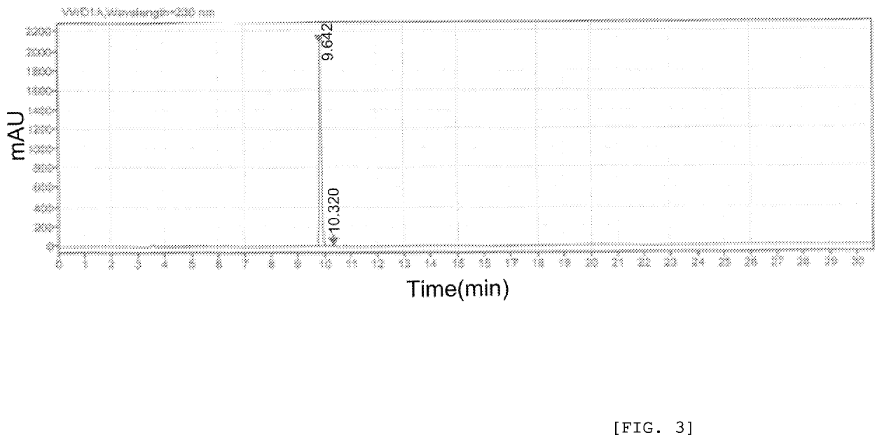

Referring to FIGS. 1 and 2, it was found that pdb1cgl_ligand has a length of about five amino-acid residues and has a relatively larger structure than GPN, PGN, Me-GPn-propyl, and Me-PGn-propyl, and seems to have more polar interaction such as hydrogen bonding.

This result contributes to having the larger Surflex-Dock score of pdb1cgl_ligand compared to the other remaining two structures. It could be seen that PGN and GPN are docking at the same position of the binding site, and the positions of asparagine commonly possessed by the two peptides almost coincide, but the position of propyl attached to C-terminal of an amino acid residue turn in the opposite direction by 180 degrees. The propyl of the Me-PGn-propyl is directed toward an open direction of a binding pocket (upward in FIG. 1), and the propyl of the Me-GPn-propyl is directed toward the inside of the binding pocket (downward in FIG. 1). It could be estimated that a difference in docking pose (conformation) of these two structures contributes to the difference in binding affinity.

Table 3 shows data on the hydrogen bond, contact, and Zn bond between ligand and receptor.

Referring to table 3, this result is shown by a significant reduction in the number of residues of amino acids which form a hydrogen bond in the case of PGN in which proline is at the N-terminus. In addition, it is estimated that GPN and PGN and the modifications GPN and PGN all interact with a Zn-residue, so as to inhibit collagenase in a distance at which they can bind to the His218, His222, and His228 amino acid residues which bind to Zn of the receptor. The inhibition of mmps was evaluated by synthesizing L-Glu-NH2 having an acyl group with a long arylalkyl side chain through a mmp inhibitor design using a peptide or non-peptide backbone having a function of chelating Zn. As a result, it was found that affinity increases on a nanomaterial basis, and a potent inhibitor of mmp needs to have a strong Zn binding function. Through molecular docking studies, it was found that glyburide binds to and interacts with a catalytic Zn residue of collagenase, and dose-dependently inhibits peptide substrate cleavage by collagenase through fluorescence measurement. These results indicate that blocking of Zn is very important for collagenase inhibition.

Through these results, it was estimated that the collagenase inhibitory activity may be greatly increased due to an increase in affinity for collagenase through molecular modification.

TABLE 3

| Ligand | H-bond | Contact | Zn-binding |
|---|---|---|---|
| 1CGL | G172, N180, L181, | S172, A184, F185, | H218, |
| (Comparative | A182, H183, Y240 | Y210, V215, E219, | H222, |
| Example 1) | | P238, S239 | H228, |
| GPN | G179, N180, L181, | S172, A184, F185, | H218, |
| (Comparative | A182, H183, Y240 | Y210, V215, E219, | H222, |
| Example 2) | | P238, S239 | H228 |
| PGN | A182, A184, E219 | L181, H183, S239, | H218, |
| (Comparative | | Y240 | H222, |
| Example 3) | | | H228 |
| Me-GPn-propyl | Asn180, L181, | R214, V215, E219, | H218, |
| (Example 6) | A182, Y237, S239 | Y240, T241 | His222, |
| | | | H228 |
| Me-PGn-propyl | Asn180, A182, | Leu181, R214, | H218, |
| (Example 7) | H183, A184, E219 | V215, S239, Y240 | H222, |
| | | | H228 |

### <Experimental Example 2> Prediction of Physical Chemistry and Cell Permeability of PGN and PGN Derivatives

Table 4 shows data on the estimation of solubility, LogP, Caco-2 cell permeability, passive permeability, and metabolic stability.

Referring to table 4, it was found that there is no significant increase in a LogP value, which is an index value of the distribution coefficient of solubility for water and octanol according to the scanning of alanine residues and the substitution of glutamine at terminal residue, but solubility is slightly decreased. It was also found that the addition of N-terminal methyl group slightly increases the rule of five violation of Lipinsky, but solubility is remarkably increased to 1000 mg/mL. It was found that the addition of n-propyl group to C-terminal amino acid increase the LogP value, an indicator of lipophilicity, compared to native peptides GPN and PGN. However, it was found that, when a methyl group is attached to the N-terminal group and an n-propyl group is attached to the C-terminal residue, solubility is significantly reduced, but the LogP value is significantly increased. It was found that peptides modified by adding a methyl group and an n-propyl group to the N-terminus and the C-terminus, respectively, significantly increase Caco-2 cell permeability compared to GPN and PGN peptides using other modification methods.

This result is because the permeation of a lipid bilayer increases as compared with polar peptide as lipophilicity increases. And it was predicted that overall cell permeability may significantly increase due to an increase in HIA index by manual transport.

And, it was predicted that the modified peptide is metabolically stable. The results of this prediction suggest that the modification of Me-GPn-propyl and Me-PGn-propyl remarkably increases the cell permeability of natural peptides GPN and PGN within the range of ensuring metabolic stability. Since percutaneous absorption increases as cell permeability increases, the embodiments of the present application have an effect of improving percutaneous absorption capacity as shown in table 4.

TABLE 4

| | | | | Caco-2 | | |
|---|---|---|---|---|---|---|
| | | | | Pe | | |
| | | Solubility | | (×10⁻⁶ | HIA | Metabolic |
| | Compound | (mg/mL) | LogP | cm/s) | (%) | stability |
| Natural | GPN | 736 | 736 | −2.26 | 0.0 | 7 |
| | (Comparative | | | | | |
| | Example 2) | | | | | |
| | PGN | 701 | 701 | −1.97 | 0.0 | 8 |
| | (Comparative | | | | | |
| | Example 3) | | | | | |
| Ala | APN | 515 | 515 | −1.98 | 0.0 | 6 |
| scanning | (Comparative | | | | | |
| | Example 4) | | | | | |
| | GAN | 482 | 482 | −2.17 | 0.0 | 9 |
| | (Comparative | | | | | |
| | Example 5) | | | | | |
| | GPA | 584 | 584 | −1.65 | 0.1 | 11 |
| | (Comparative | | | | | |
| | Example 6) | | | | | |
| | AGN | 391 | 391 | −2.17 | 0.0 | 9 |
| | (Comparative | | | | | |
| | Example 7) | | | | | |
| | PAN | 475 | 475 | −1.79 | 0.0 | 6 |
| | (Comparative | | | | | |
| | Example 8) | | | | | |
| | PGA | 544 | 544 | −1.16 | 0.2 | 12 |
| | (Comparative | | | | | |
| | Example 9) | | | | | |
| Gln | GPQ | 455 | 455 | −2.29 | 0.0 | 6 |
| substituent | (Comparative | | | | | |
| | Example 10) | | | | | |
| | PGQ | 558 | 558 | −2.08 | 0.0 | 6 |
| | (Comparative | | | | | |
| | Example 11) | | | | | |
| N-terminal | Me-GPN | 1000 | 1000 | −1.71 | 0.0 | 1 |
| Methyl | (Example 3) | | | | | |
| | Me-PGN | 1000 | 1000 | −2.09 | 0.0 | 1 |
| | (Example 2) | | | | | |
| C-terminal | GPn-propyl | 1000 | 1000 | −1.16 | 0.1 | 13 |
| n-propyl | (Example 4) | | | | | |
| | PGn-propyl | 1000 | 1000 | −0.81 | 0.0 | 13 |
| | (Example 5) | | | | | |
| Methyl and | Me-GPn-propyl | 19.7 | 19.7 | −0.81 | 0.9 | 53 |
| n-propyl | (Example 6) | | | | | |
| | Me-PGn-propyl | 139 | 139 | −0.52 | 1.0 | 68 |
| | (Example 7) | | | | | |

### <Experimental Example 3> Comparison of Collagenase Inhibitory Activity of PGN (Comparative Example 3) and Derivative Thereof

Collagenase inhibitory activity was performed by purchasing EnzChek® Gelatinase/Collagenase Assay Kit (250-2000 Assays) reagent from Invitrogen. First, DQ collagen stock solution (1 mg/ml) was prepared by adding 1.0 ml of DDW to 1 mg DQ collagen vial. The 18 ml of DDW was added to 2 ml of 10× reaction buffer, so as to dilute reaction buffer. Then, a collagenase enzyme reagent was prepared, and diluted with reaction buffer to a final concentration of 0.2 U/ml as working solution. Three samples of 50 [symbol] each were prepared in a 96-well plate. The 30 [symbol] of reaction buffer was added to 20 [symbol] of DQ collagen and sample blank, and 30 [symbol] of working solution was added to a sample, which were then left in a light-blocked state at room temperature for about one to two hours, and then fluorescence intensity was measured at excitation wavelength 485 nm and emission wavelength 535 nm using ELISA plate reader (VICTOR X3™, PerkinElmer, US).

As a positive control, copper peptide (Comparative Example 12), which is a representative peptide widely used as a raw material for functional cosmetics, was prepared at the same concentration and used. Copper peptide has excellent skin regeneration ability and thus is used as a raw material to suppress skin aging by promoting wound healing, strengthening skin elasticity, and increasing subcutaneous fat layers.

$$Collagenase activity inhibition rate ( \% ) = 100 - \frac{b - {b}^{′}}{a - {a}^{′}} \times 100 \qquad [ Equation 1 ]$$

$$a : Absorbance after reaction of blank sample solution \\ b : Absorbance after reaction of sample solution \\ {a}^{′} , {b}^{′} : Absorbance measured by replacing collagenase with buffer$$

TABLE 5

| | | Collagenase |
|---|---|---|
| | Concentration | inhibitory |
| Sample name | (mg/ [symbol] ) | activity (%) |
| PGN | 0.05 | 1.6 ± 3.3 |
| (Comparative | 0.1 | 4.7 ± 3.9 |
| Example 3) | 0.5 | 11.2 ± 6.2 |
| | 1 | 13.1 ± 1.7 |
| Me-PGN | 0.05 | 3.6 ± 2.4 |
| (Example 2) | 0.1 | 15.7 ± 1.4 |
| | 0.5 | 15.7 ± 7.4 |
| | 1 | 46.3 ± 4.8 |
| PGN-OPr | 0.05 | 5.7 ± 3.1 |
| (Example 1) | 0.1 | 14.3 ± 5.2 |
| | 0.5 | 67.1 ± 0.8 |
| | 1 | 98.7 ± 0.2 |
| Pal-PGN | 0.05 | 7.4 ± 2.2 |
| (Example 8) | 0.1 | 11 ± 5.4 |
| | 0.5 | 21.3 ± 3.2 |
| | 1 | 24.1 ± 2.4 |
| Copper Peptide | 1 | 14.6 ± 1.9 |
| (Comparative | | |
| Example 12) | | |

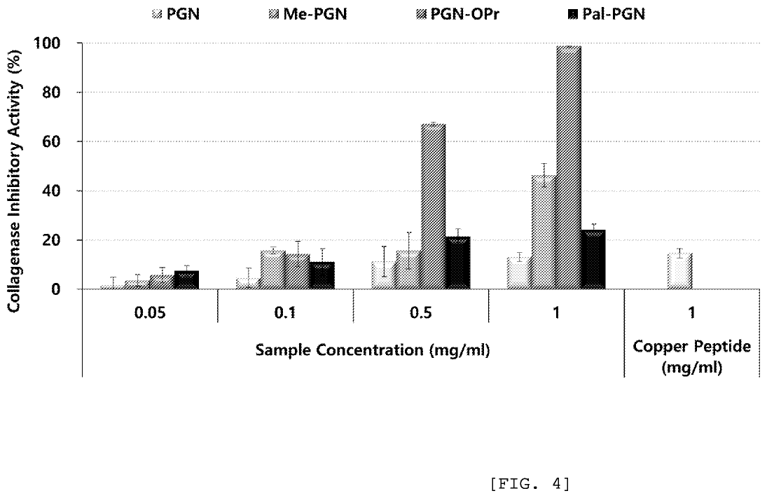

The collagenase inhibitory activity results are shown in table 5 and FIGS. 4 to 6. In all samples, it was found that the collagenase inhibitory activity increases as the concentration increases. It can be seen that the sample is concentration-dependent.

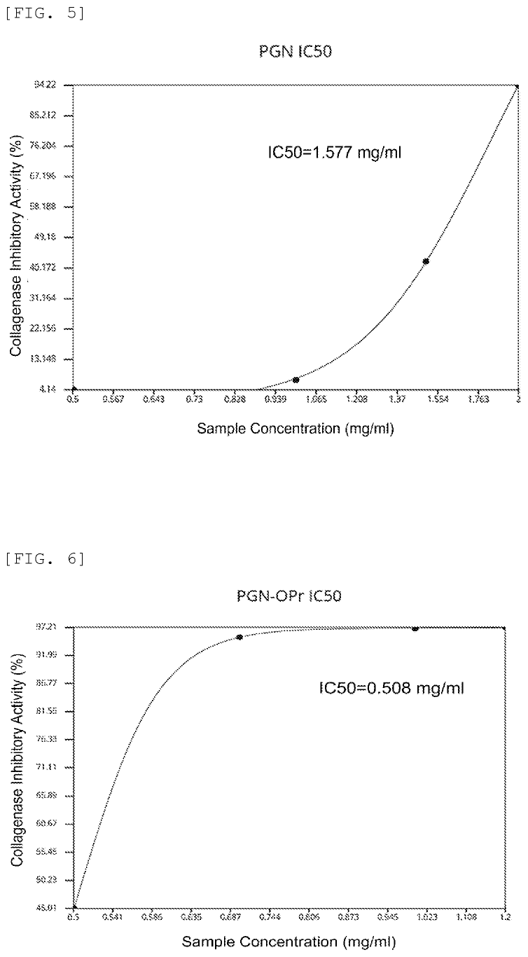

It was found that PGN-Opr has the highest inhibitory activity at a concentration of 1 mg/ml with PGN (Comparative Example 3) of 13.1%, Me-PGN (Example 2) of 46.3%, PGN-OPr (Example 1) of 98.7%, and Pal-PGN (Example 8) of 24.1%, respectively. Copper peptide (Comparative Example 12) used as a control at the same concentration showed the inhibitory activity of about 14.6%, indicating that PGN-OPr (Example 1) shows about seven times higher activity than that of Copper peptide (Comparative Example 12). In addition, referring to FIGS. 5 and 6, PGN (Comparative Example 3) had the collagenase inhibitory activity IC50 of 1.577 mg/ml, but PGN-OPr (Example 1), the PGN derivative, had the IC50 of 0.508 mg/ml, indicating that the activity of the derivative is increased three times or more.

### <Experimental Example 4> Comparison of Elastase Inhibitory Activity of PGN (Comparative Example 3) and Derivative Thereof

Elastase inhibitory activity was performed by purchasing EnzChek® Elastase Assay Kit (600 Assays) reagent from Invitrogen. First, DQ elastin stock solution (1 mg/ml) was prepared by adding 1.0 ml of DDW to 1 mg DQ elastin vial. The 18 ml of DDW was added to 2 ml of 10× reaction buffer, so as to dilute reaction buffer. Then, an elastase enzyme reagent was prepared, and diluted with reaction buffer to a final concentration of 0.2 U/ml as working solution. Three samples of 50 [symbol] each were prepared in a 96-well plate. The 50 [symbol] of reaction buffer was added to 50 [symbol] of DQ elastin and sample blank, and 50 [symbol] of working solution was added to a sample, which were then left in a light-blocked state at room temperature for about one hour, and then fluorescence intensity was measured at excitation wavelength 505 nm and emission wavelength 515 nm using ELISA plate reader (VICTOR X3™, PerkinElmer, US).

Copper peptide (Comparative Example 12) and EGCg (Comparative Example 13), which is widely used as a raw material for functional cosmetics, were prepared at the same concentration and used as a positive control.

EGCg (epigallocatechin gallate) is a kind of polyphenol, which is an extract of green tea leaves, and is used as a cosmetic raw material for anti-oxidation, inflammation reduction, and wrinkle alleviation, and Copper peptide is used as a raw material for promoting wound healing, strengthening skin elasticity, and increasing subcutaneous fat layer to suppress skin aging due to excellent skin regeneration ability.

$$Elastase activity inhibition rate ( \% ) = 100 - \frac{b - {b}^{′}}{a - {a}^{′}} \times 100 \qquad [ Equation 2 ]$$

$$a : Absorbance after reaction of blank sample solution \\ b : Absorbance after reaction of sample solution \\ {a}^{′} , {b}^{′} : Absorbance measured by replacing elastase with buffer$$

TABLE 6

| | | Elastase |
|---|---|---|
| | Concentration | Inhibitory |
| Sample name | (mg/ [symbol] ) | Activity (%) |
| PGN | 0.02 | 1.8 ± 0.6 |
| (Comparative | 0.04 | 2.8 ± 1.2 |
| Example 3) | 0.06 | 6.1 ± 1.9 |
| | 0.1 | 7.5 ± 3.2 |
| Me-PGN | 0.02 | 5.2 ± 1.7 |
| (Example 2) | 0.04 | 6.9 ± 1.8 |
| | 0.06 | 9.9 ± 1.5 |
| | 0.1 | 11.2 ± 1.1 |
| PGN-OPr | 0.02 | 17.3 ± 1.1 |
| (Example 1) | 0.04 | 27.5 ± 2.5 |
| | 0.06 | 34.2 ± 0.5 |
| | 0.1 | 77.6 ± 2.8 |
| Pal-PGN | 0.02 | 0.3 ± 0.9 |
| (Example 8) | 0.04 | 1.2 ± 2.8 |
| | 0.06 | 1.5 ± 2 |
| | 0.1 | 5.2 ± 2.9 |
| EGCg | 0.1 | 66.1 ± 4.4 |
| (Comparative | | |
| Example 13) | | |
| Copper peptide | 0.1 | 18.8 ± 3.5 |
| (Comparative | | |
| Example 12) | | |

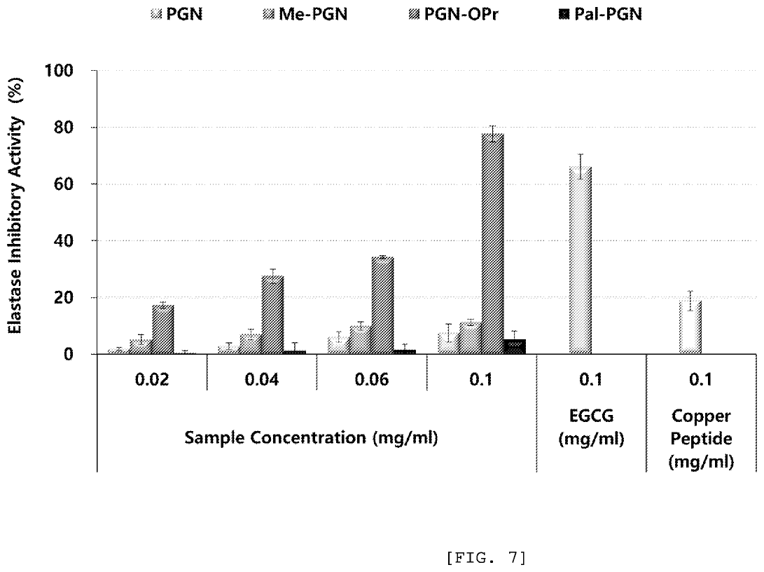

The elastase inhibitory activity results are shown in table 6 and FIGS. 7 to 9. In all samples, it was found that the elastase inhibitory activity increases as the concentration increases. It can be seen that the sample is concentration-dependent.

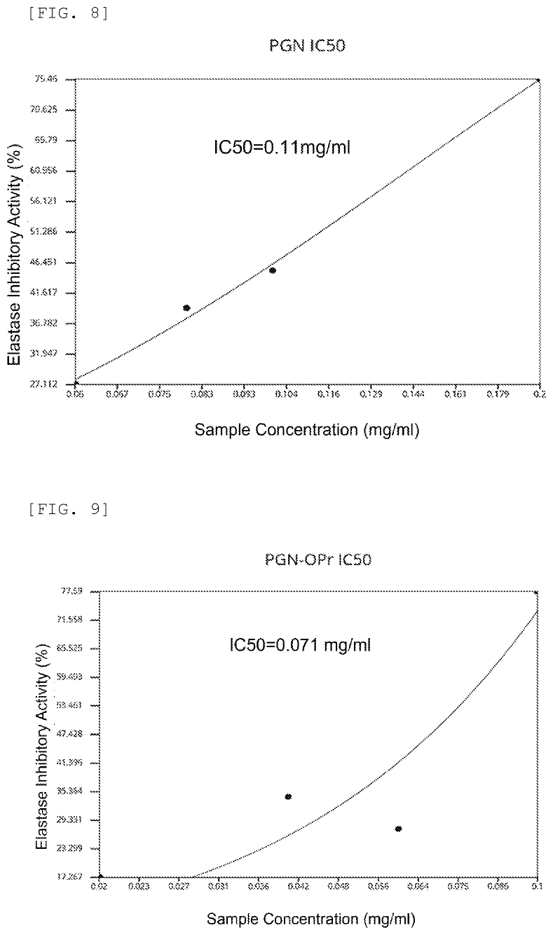

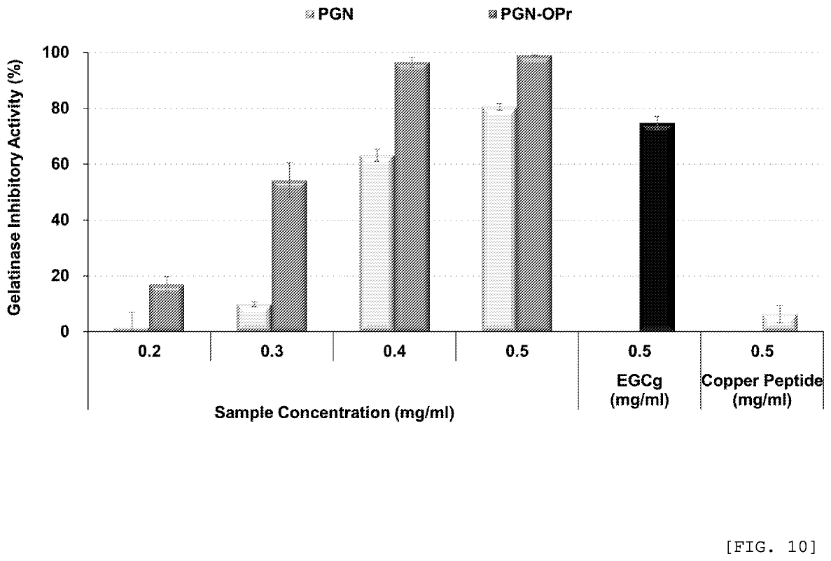

At a concentration of 1 mg/ml, the inhibitory activity was presented as PGN of 7.5%, Me-PGN of 11.2%, and PGN-OPr of 77.6%, so the PGN-Opr had the highest inhibitory activity. At the same concentration, the comparative controls EGCg and Copper peptide were 66.1% and 18.8%, respectively. PGN-Opr was more active than EGCg. Referring to FIGS. 8 and 9, PGN (Comparative Example 3) had the elastase inhibitory activity IC50 of 0.110 mg/ml, but PGN-OPr (Example 1), the PGN derivative, had the IC50 of 0.071 mg/ml, indicating a more increase in the activity of the derivative.

### <Experimental Example 5> Comparison of Gelatinase Inhibitory Activity of PGN (Comparative Example 3) and Derivative Thereof

Gelatinase is an enzyme which plays a major role in promoting skin aging by more finely degrading collagen fragments digested by MMP-1 with MMP-2, and wrinkle reduction activity was determined by measuring the inhibitory activity of the enzyme.

Gelatinase inhibitory activity was performed by purchasing EnzChek® Gelatinase/Collagenase Assay Kit (250-2000 Assays) reagent from Invitrogen. First, DQ gelatin stock solution (1 mg/ml) was prepared by adding 1.0 ml of DDW to 1 mg DQ gelatin vial. The 18 ml of DDW was added to 2 ml of 10× reaction buffer, so as to dilute reaction buffer. Then, a gelatinase enzyme reagent was prepared, and diluted with reaction buffer to a final concentration of 0.2 U/ml as working solution. Three samples of 50 [symbol] each were prepared in a 96-well plate. The 30 [symbol] of reaction buffer was added to 20 [symbol] of DQ gelatin and sample blank, and 30 [symbol] of working solution was added to a sample, which were then left in a light-blocked state at room temperature for about one to two hours, and then fluorescence intensity was measured at excitation wavelength 485 nm and 535 nm using ELISA plate reader (VICTOR X3™, PerkinElmer, US).

$$Gelatinase activity inhibition rate ( \% ) = 100 - \frac{b - {b}^{′}}{a - {a}^{′}} \times 100 \qquad [ Equation 3 ]$$

$$a : Absorbance after reaction of blank sample solution \\ b : Absorbance after reaction of sample solution \\ {a}^{′} , {b}^{′} : Absorbance measured by replacing gelatinase with buffer$$

TABLE 7

| | Concentration | Gelatinase inhibitory |
|---|---|---|
| Sample name | (mg/ [symbol] ) | activity (%) |
| PGN | 0.2 | 1.5 ± 5.5 |
| (Comparative | 0.3 | 9.8 ± 0.8 |
| Example 3) | 0.4 | 63.1 ± 2.1 |
| | 0.5 | 80.5 ± 1.2 |
| PGN-OPr | 0.2 | 16.9 ± 2.7 |
| (Example 1) | 0.3 | 54.2 ± 6.3 |
| | 0.4 | 96.4 ± 1.8 |
| | 0.5 | 98.9 ± 0.1 |
| EGCg | 0.5 | 74.7 ± 2.3 |
| (Comparative | | |
| Example 13) | | |
| Copper peptide | 0.5 | 6.2 ± 3.1 |
| (Comparative | | |
| Example 12) | | |

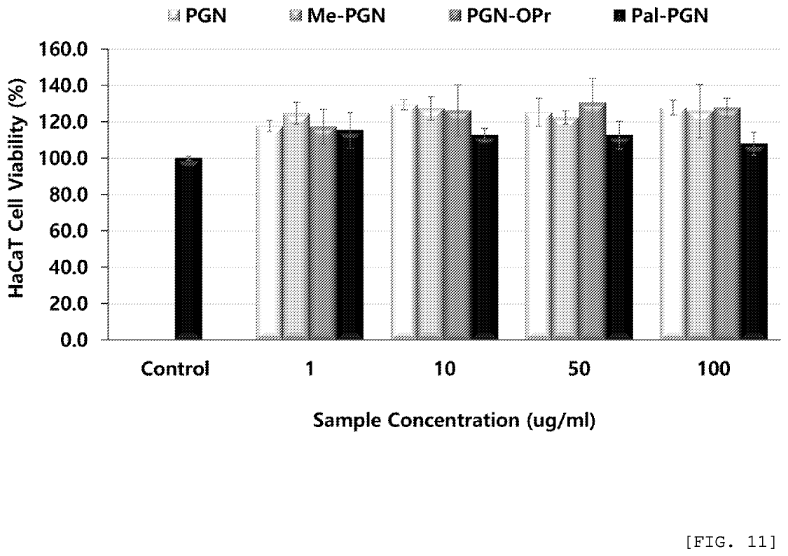

The gelatinase inhibitory activity results are shown in table 7 and FIG. 10. In all samples, it was found that the gelatinase inhibitory activity significantly increases as the concentration increases.

PGN-Opr (Example 1) had a gelatinase inhibitory activity of 98.9% at 0.5 mg/ [symbol] , which was higher than that of Comparative Examples. EGCg (Comparative Example 13) and Copper peptide (Comparative Example 12) measured at the same concentration were inhibited at 74.7% and 6.2%, respectively.

PGN-OPr was confirmed to have very excellent efficacy with gelatinase inhibitory activity about 1.3 times higher than that of EGCg and 15 times higher than that of Copper peptide. PGN-Opr had very high efficacy of inhibiting the activity of enzymes which induce wrinkle formation, suggesting that PGN-Opr is a very suitable material for use as a wrinkle prevention material.

### <Experimental Example 6> Safety Evaluation of PGN (Comparative Example 3) and Derivative Thereof Through Toxicity Test of Human Keratinocyte (HaCaT Cell, Human Skin Epidermal Cells

HaCaT cells, skin epidermal cells (derived from human), were used. The medium was DMEM low (SH30021.01, HyClone™, Logan, UT, USA) medium containing 10% fetal bovine serum (FBS, Lonza, Valais, Switzerland) medium. The cell lines were cultured in an incubator adjusted to 95% humidity, 5% CO2 and 37° C., and at this time, a medium antibiotic (Penicillin streptomycin, Gibco, Calif., USA) was used to suppress contamination or proliferation of microorganisms. When the cells covered the dish about 80%, the cells were washed with phosphated-buffered saline-EDTA (PBS-EDTA), and then the cells attached to the bottom of the dish were detached by treating with 1 ml of 0.05% trypsin EDTA, and subjected to subculture. The medium was exchanged every 48 hours to culture the cells.

Cell viability was measured using MTS (CellTiter 96® AQueous One Solution Cell Proliferation Assay, Promega, USA) reagent. The cells were divided at a concentration of 1×10⁴ cells/well in a 96-well plate and then stabilized for 24 hours in an incubator controlled to 95% humidity, 5% CO₂ and 37° C. After that, the samples were treated for each concentration (table 1) and cultured for 24 hours in an incubator controlled to 95% humidity, 5% CO₂ and 37° C. After the medium was removed, 9 ml of medium (DMEM low, free FBS) was added to 1 ml of the reagent and diluted, and then 100 ul of the diluted solution was treated per well. After the reaction at 37° C. for 60 minutes, absorbance was measured at 490 nm with microplate reader (Molecular Devices, VersaMax ELISA Microplate Reader, USA). Cell viability was represented by absorbance of a sample-treated group compared to a control group not treated with the sample.

TABLE 8

| | Concentration | Cell viability |
|---|---|---|
| Sample name | (μg/ [symbol] ) | (%) |
| Control | — | 100 ± 1.2 |
| PGN | 1 | 117.7 ± 3.1 |
| (Comparative | 10 | 129.3 ± 2.8 |
| Example 3) | 50 | 125.2 ± 7.6 |
| | 100 | 127.9 ± 4.1 |
| Me-PGN | 1 | 124.7 ± 6.1 |
| (Example 2) | 10 | 127.4 ± 6.5 |
| | 50 | 122.5 ± 3.7 |
| | 100 | 125.9 ± 14.8 |
| PGN-OPr | 1 | 117.3 ± 9.6 |
| (Example 1) | 10 | 126.1 ± 14.1 |
| | 50 | 130.4 ± 13.3 |
| | 100 | 127.7 ± 5.3 |
| Pal-PGN | 1 | 115.2 ± 10 |
| (Example 8) | 10 | 112.5 ± 3.7 |
| | 50 | 112.7 ± 7.7 |
| | 100 | 107.8 ± 6.5 |

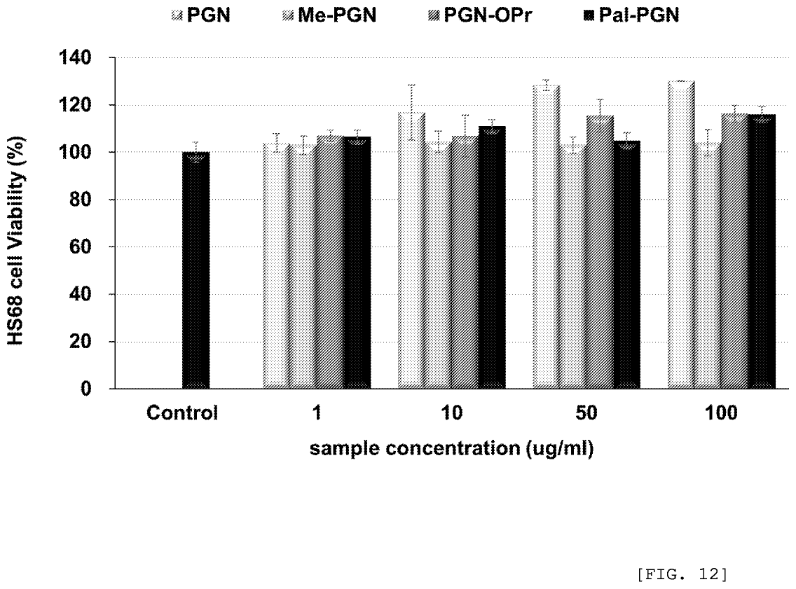

The results of the HaCaT cytotoxicity test of PGN (Comparative Example 3), Me-PGN (Example 2), PGN-OPr (Example 1), and Pal-PGN (Example 8) are shown in table 8 and FIG. 11. All samples were found to be non-toxic at a concentration of 1-100 μg/ml.

### <Experimental Example 7> Safety Evaluation of PGN (Comparative Example 3) and Derivative Thereof Through Toxicity Test of Skin Fibroblasts (HS68 Cells

Skin fibroblast HS68 cells were used. The medium was DMEM high glucose (SH30243.01, HyClone™, Logan, UT, USA) medium containing 10% fetal bovine serum (FBS, Lonza, Valais, Switzerland) medium. The cell lines were cultured in an incubator adjusted to 95% humidity, 5% CO₂ and 37° C., and at this time, a medium antibiotic (Penicillin streptomycin, Gibco, Calif., USA) was used to suppress contamination or proliferation of microorganisms.

Cell viability was measured using MTS (CellTiter 96® AQueous One Solution Cell Proliferation Assay, Promega, USA) reagent. The cells were divided at a concentration of 1×10⁴ cells/well in a 96-well plate and then stabilized for 24 hours in an incubator controlled to 95% humidity, 5% CO₂ and 37° C. After that, the samples were treated for each concentration and cultured for 24 hours in an incubator controlled to 95% j humidity, 5% CO₂ and 37° C. After the medium was removed, 9 ml of medium (DMEM high, free FBS) was added to 1 ml of the reagent and diluted, and then 100 ul of the diluted solution was treated per well. After the reaction at 37° C. for 60 minutes, absorbance was measured at 490 nm with microplate reader (Molecular Devices, VersaMax ELISA Microplate Reader, USA). Cell viability was represented by absorbance of a sample-treated group compared to a control group not treated with the sample.

TABLE 9

| | Concentration | Cell viability |
|---|---|---|
| Sample name | (μg/ [symbol] ) | (%) |
| Control | — | 100 ± 4.2 |
| PGN | 1 | 103.9 ± 3.9 |
| (Comparative | 10 | 116.8 ± 11.5 |
| Example 3) | 50 | 128.3 ± 2.2 |
| | 100 | 130.1 ± 0.1 |
| Me-PGN | 1 | 102.9 ± 3.9 |
| (Example 2) | 10 | 104.4 ± 4.5 |
| | 50 | 103 ± 3.4 |
| | 100 | 104.1 ± 5.6 |
| PGN-OPr | 1 | 106.9 ± 2.4 |
| (Example 1) | 10 | 106.7 ± 8.9 |
| | 50 | 115.4 ± 6.9 |
| | 100 | 116.3 ± 3.6 |
| Pal-PGN | 1 | 106.4 ± 2.9 |
| (Example 8) | 10 | 110.9 ± 2.8 |
| | 50 | 104.8 ± 3.3 |
| | 100 | 115.9 ± 3.3 |

The results of the HS68 cytotoxicity test of PGN (Comparative Example 3), Me-PGN (Example 2), PGN-OPr (Example 1), and Pal-PGN (Example 8) are shown in table 8 and FIG. 12. No toxicity was observed at the concentration of 1-100 μg/ml in all samples.

Hereinafter, an example of the formulation of the cosmetic composition according to the present application will be described, but various formulations other than the following examples may be used, and this is not intended to limit the present application, but is intended to be specifically described.

### <Formulation Example 1> Softening Lotion (Skin Lotion

A softening lotion was prepared by a conventional method according to the composition shown in table 10 below.

TABLE 10

| Ingredient | Content (wt %) |
|---|---|
| Example 1 | 0.25 |
| Glycerin | 3.5 |
| Oleyl alcohol | 1.5 |
| Ethanol | 5.5 |
| Polysorbate 80 | 3.2 |
| Carboxylic vinyl polymer | 1.0 |
| Butylene glycol | 2.0 |
| Propylene glycol | 2.0 |
| Preservative, perfume | Suitable amount |
| Purified water | Remaining amount |
| Sum | 100 |

### <Formulation Example 2> Nourishing Lotion (Milk Lotion

A nourishing lotion was prepared by a conventional method according to the composition shown in table 11 below.

TABLE 11

| Ingredient | Content (wt %) |
|---|---|
| Example 1 | 0.25 |
| Glycerin | 3.0 |
| Butylene glycol | 3.0 |
| Propylene glycol | 3.0 |
| Carboxyvinyl polymer | 0.1 |
| Wax | 4.0 |
| Polysorbate 60 | 1.5 |
| Caprylic/Capric triglyceride | 5.0 |
| Squalane | 5.0 |
| Sorbitan sesquioleate | 1.5 |
| Cetearyl alcohol | 1.0 |
| Triethanolamine | 0.2 |
| Preservative, perfume | Suitable amount |
| Purified water | Remaining amount |
| Sum | 100 |

### <Formulation Example 3> Nourishing Cream

A nourishing cream was prepared by a conventional method according to the composition shown in table 12 below.

TABLE 12

| Ingredient | Content (wt %) |
|---|---|
| Example 1 | 0.25 |
| Glycerin | 3.5 |
| Butylene glycol | 3.0 |
| Flowing paraffin | 7.0 |
| Beta-glucan | 7.0 |
| Carbomer | 0.1 |
| Caprylic/Capric triglyceride | 3.0 |
| Squalane | 5.0 |
| Cetearyl glucoside | 1.5 |
| Sorbitan stearate | 0.4 |
| Polysorbate 60 | 1.2 |
| Triethanolamine | 0.1 |
| Preservative, perfume | Suitable amount |
| Purified water | Remaining amount |
| Sum | 100 |

Although the present application has been described in detail with reference to Preparation Examples, Examples, Comparative Examples, and Experimental Examples, the present application is not limited to above Preparation Examples, Examples, and Experimental Examples, and various modifications and changes may be made by those skilled in the art within the technical spirit and scope of the present application.

While specific portions of the present invention have been described in detail above, it is apparent to those skilled in the art that such detailed descriptions are set forth to illustrate exemplary embodiments only, but are not construed to limit the scope of the present invention. Thus, it should be understood that the substantial scope of the present invention is defined by the accompanying claims and equivalents thereto.

1. A peptide derivative represented by formula 1 below or a pharmaceutically acceptable salt thereof: [Formula 1] R₁-L-R₂ wherein, L is Gly-Pro-Asn (GPN) or Pro-Gly-Asn (PGN), R₁ is hydrogen, C₁-C₆ alkyl group, C₁-C₆ alkoxy group, palmitoyl group, lauroyl group, myristoyl group, stearoyl group, arachidoyl group or linoleoyl group, R₂ is a hydroxy group, C₁-C₆ alkyl group or C₁-C₆ alkoxy group, and when R is hydrogen, R₂ is not a hydroxy group.

2. The peptide derivative or the pharmaceutically acceptable salt thereof of claim 1, wherein above formula 1 is represented by formula 2 and formula 3: 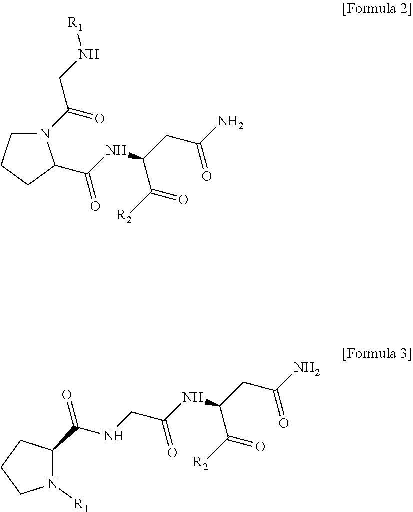
wherein, in above formula 2 or 3, R₁ is hydrogen, CH₃ or a palmitoyl group, and R₂ is a hydroxy group, C₃H₇ or O—C₃H₇, and when R₁ is hydrogen, R₂ is not a hydroxy group.

3. The peptide derivative or the pharmaceutically acceptable salt thereof of claim 2, wherein above formula 2 is any one of formulas 4 to 8:

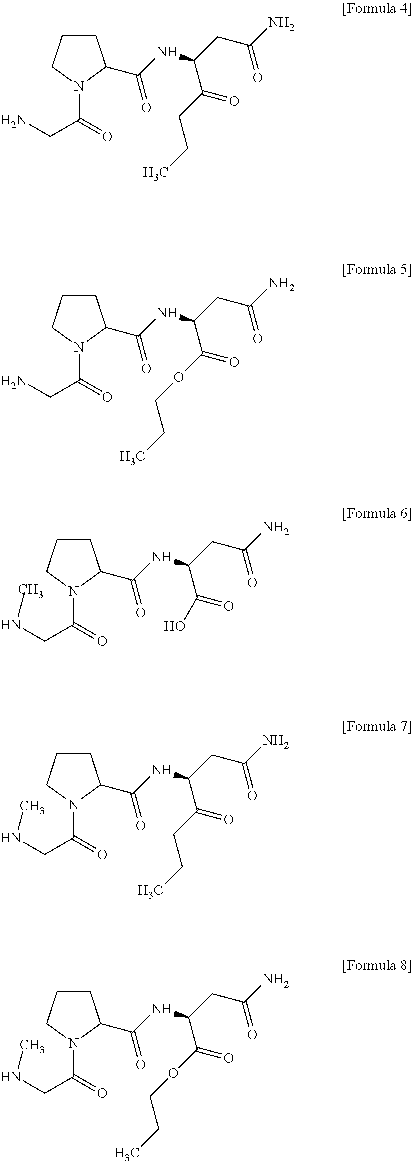

4. The peptide derivative or the pharmaceutically acceptable salt thereof of claim 2, wherein above formula 3 is any one of formulas 9 to 13:

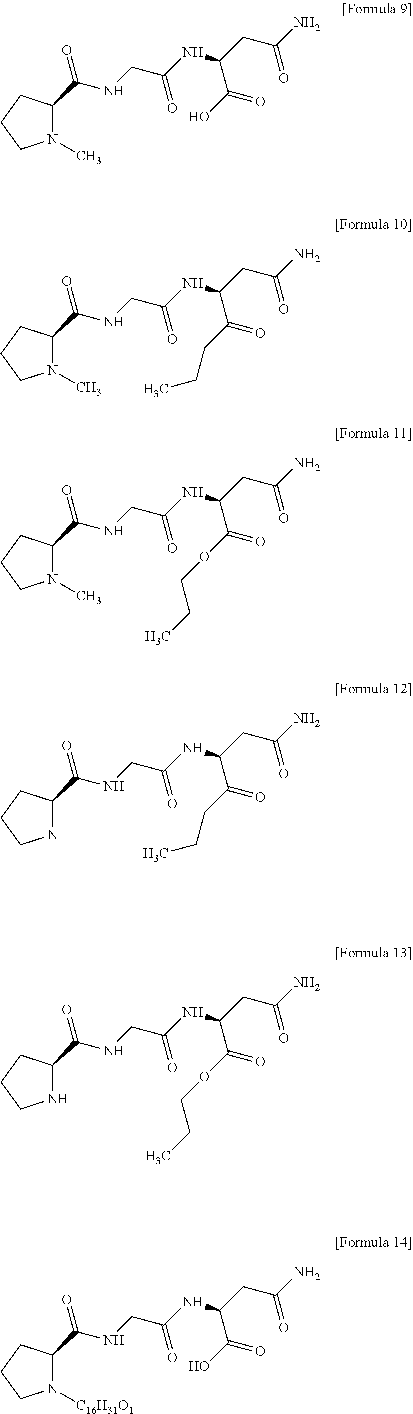

5. A cosmetic composition comprising: as an active ingredient, a peptide derivative according to any one of claims 1 to 4; or a pharmaceutically acceptable salt thereof.

6. The cosmetic composition of claim 5, wherein the cosmetic composition is for alleviating skin aging, which inhibits activity and production of collagenase.

7. The cosmetic composition of claim 5, wherein the cosmetic composition is for reducing skin wrinkles or improving skin elasticity.

8. The cosmetic composition of claim 5, wherein the cosmetic composition has improved percutaneous absorbing capacity.

9. A pharmaceutical composition comprising: as an active ingredient, a peptide derivative according to any one of claims 1 to 4; or a pharmaceutically acceptable salt thereof, wherein a pharmaceutical composition inhibits activity and production of collagenase.

10. A health functional food composition comprising: as an active ingredient, a peptide derivative according to any one of claims 1 to 4; or a pharmaceutically acceptable salt thereof, wherein a health functional food composition inhibits activity and production of collagenase.

11. A use of a peptide derivative according to any one of claims 1 to 4 or a pharmaceutically acceptable salt thereof for preparing a drug for inhibiting activity and production of collagenase.
